(19) United States
(12) Patent Application Publication
(10) Pub. No.: US 2013/0209419 A1
(43) Pub. Date: Aug. 15, 2013

(54) Methods of Engraftment

(71) Applicant: Therapure Biopharma Inc., (US)

(72) Inventors: Kathryn E. Matthews, Toronto (CA); David N. Bell, Oakville (CA)

(73) Assignee: Therapure Biopharma Inc., Mississauga (CA)

(21) Appl. No.: 13/768,830

(22) Filed: Feb. 15, 2013

Related U.S. Application Data
(60) Provisional application No. 61/599,136, filed on Feb. 15, 2012.

(51) Int. Cl. A61K 38/02

(52) U.S. Cl. CPC A61K 38/02
USPC 424/93.7; 514/13.5

(57) ABSTRACT

Methods and compositions for improving the engraftment of stem cells are described. The method involves administering an effective amount of hemoglobin. The methods and compositions are useful in stimulating hematopoiesis and may be used to boost human hematopoietic re-engraftment for use in transplant recipients or in patients with bone marrow injury.

This application claims the benefit under 35 USC §119(e) from U.S. Provisional patent application serial number 61/599,136, filed Feb. 15, 2012, which is incorporated herein by reference.

### FIELD

The present disclosure relates to methods and compositions for improving the engraftment of human stem cells in a mammal.

### BACKGROUND

Hematopoiesis is defined as the production and development of blood cells, including erythrocytes, granulocytes, monocytes, macrophages, esoinophils, basophils, megakaryocytes, B cells and T cells (Wintrobe, 1999). Hematopoiesis occurs as the result of the proliferation and differentiation of hematopoietic stem cells. Hematopoietic stem cells are pluripotent cells which can give rise to the multiple cell lineages found in the blood. Hematopoietic stem cells reside in the bone marrow and their growth, proliferation and differentiation are influenced by both hematopoietic growth factors and the stromal cells within the bone marrow. Stem cells are believed to normally reside in a quiescent nondividing state until stimulated by specific growth factors whereupon they divide and give rise to highly proliferative progenitor cells committed to the production of blood cells of one or more lineages, such as the erythroid, myeloid or lymphoid lineages.

Certain clinical disorders, termed cytopenias, are characterized by the decreased level of a specific cell type in the circulating blood. For example neutropenia is a disorder whereby there is a diminished level of circulating neutrophils. This disorder can be treated by GM-CSF or G-CSF, two different hematopoietic growth factors. However, administration of these growth factors is often associated with a high incidence of adverse side effects. For example, the administration of G-CSF after allogeneic bone marrow transplantation may result in dyspnea, chest pain, nausea, hypoxemia, diaphoresis, anaphylaxis, syncope and flushing (Khoury et al, 2000).

Neutropenia is also associated with AIDS and is currently treated with growth factors (Dubreuil-Lemaire et al, 2000). There are also forms of severe congenital neutropenia (Dale et al, 2000) in which a small percentage of the patients are refractory to the administration of growth factors.

In order to be able to conduct in vivo studies on human hematopoiesis without using human subjects, mice which have been partially engrafted with human hematopoietic stem cells are often used as a model. These mice hosts are termed SCID (severe combined immuno-deficient) mice that have been bred to lack a functional immune system and are deficient in mature lymphocytes and NK cells. One such strain of these mice, the NOD.Cg-Prkdc^{scid} IL2rgt^{m1Wjl}/SzJ, is referred to as the NSG (NOD SCID gamma) mouse. The immune deficiencies created in these animals allow for the partial engraftment and to some extent, the proliferation and differentiation of transplanted human hematopoietic stem cells (CD34⁺ cells derived from human bone marrow, for example). However, these studies are often hampered by poor engraftment of the humans cells within the bone marrow of the host mice with the resultant loss of the transplanted human stem cells in the animals, low levels of human hemaₜopoiesis (often determined by measuring the level of human CD45⁺ cells) or large variations in the degree of human hematopoiesis amongst the same animals within a study.

In view of the foregoing, there is a need in the art to develop improved methods for increasing the number of blood cells in a mammal through improved/enhanced hematopoietic engraftment of transplanted hematopoietic cells. There is also a need to improve animal models for use in studying human hematopoiesis and for developing therapies for disorders requiring the stimulation of hematopoiesis.

### SUMMARY

The present inventors have determined that administration of hemoglobin can stimulate the growth, proliferation and differentiation of both erythroid and myeloid progenitors in vivo, leading to increased blood cell production. In particular, the inventors have shown that administering hemoglobin to an immunocompromised mouse that has been transplanted with human CD34⁺ stem cells increased the engraftment of the human cells and increased the number of bone marrow progenitor cell colonies detected.

Accordingly, the present disclosure provides a method of improving the engraftment of a stem cell in an animal, comprising administering an effective amount of hemoglobin to the animal.

The present disclosure also includes pharmaceutical compositions comprising an effective amount of hemoglobin in admixture with a suitable diluent or carrier for use in enhancing stem cell engraftment in a mammal.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art of this detailed description.

### BRIEF DESCRIPTION OF THE DRAWINGS

FIG. 1 is a graph showing the percent of human bone borrow CD45⁺ cells post treatment with hemoglobin or a PBS control.

FIG. 2 is a graph showing the percent of human bone marrow CD34⁺ cells post treatment with hemoglobin or a PBS control.

FIG. 3 is a graph showing the number of colony-forming cells/plate.

### DETAILED DESCRIPTION

The inventors used a humanized SCID mouse as a model for stimulating hematopoiesis in vivo. These severely immunocompromised mice were transplanted with purified human CD34⁺ (as determined by >30% human CD45⁺ cells in the peripheral blood). Purified human hemoglobin (2 mg/mouse) was administered every 4 days over a 12 day period. At day 14, bone marrow cells were removed and examined by flow cytometry and pooled and enriched for CD34⁺ cells which were then plated in colony-forming assays. Surprisingly, the number of human CD34⁺ cells increased (to 7%) with the administration of hemoglobin. A similar pattern was observed for human CD45⁺ cells and the total number of colony-forming cells detectable. These data indicate that hemoglobin has direct and measurable effect on human hematopoietic cell engraftment in vivo.

Accordingly, the present disclosure provides a method of improving the engraftment of a stem cell in an animal, comprising administering an effective amount of hemoglobin to the animal.

Improved engraftment can be measured by the increase in human CD45⁺ cells. CD45 is a pan-leukocytic marker and is expressed on all hematopoietic cells with the exception of platelets and enucleated red blood cells. In one embodiment, the CD45⁺ cells are increased by at least 10% as compared to the levels in an engrafted animal that did not receive hemoglobin. In another embodiment, the CD45⁺ cells are increased by at least 20% as compared to the levels in an engrafted animal that did not receive hemoglobin. In yet another embodiment, the CD45⁺ cells are increased by at least 30% as compared to the levels in an engrafted animal that did not receive hemoglobin.

The term “stem cell” as used herein means a cell that is capable of differentiating into any cell including hematopoietic cells in an animal. The term includes hematopoietic stem cells and hematopoietic progenitor cells.

Preferably, the stem cell is a CD34⁺ stem cell. CD34⁺ cells are traditionally considered “stem” cells in that they are capable of both self-renewal and re-populating an individual with cells from all hematopoietic lineages. CD34⁺ cells are further delineated into sub-populations by the co-expression of other markers, such as CD38, and the ability of these sub-populations to re-engraft and repopulate the hematopoietic system (eg., Henan et al., 2001). The stem cell may also express the CD163 receptor.

The CD34⁺ stem cell will be purified prior to administration or transplantation into the animal. CD34⁺ cells can be purified using techniques known in the art. For transplantation into humans they may be autologous stem cells or obtained from a suitable donor.

The present disclosure also provides a method of improving the engraftment of a stem cell in an animal comprising (a) transplanting stem cells in the animal and (b) administering an effective amount of hemoglobin to the animal.

The hemoglobin can be administered concurrently or after the stem cell transplant. Preferably, the hemoglobin is administered 1 to 10 days, more preferably 1 to 5 days or 1 to 2 days after the stem cells have been administered to the animal.

The hemoglobin can be any purified human hemoglobin that is pharmaceutically acceptable. The hemoglobin is preferably human hemoglobin. In one embodiment, a HemA_{zerc}, solution is used. HemA_{zero} is a solution of purified human hemoglobin. The hemoglobin was derived from human red blood cells collected from screened donors at a licensed and accredited facility and intended for transfusion. All units of red blood cells used in the process have been tested and found non-reactive for HIV-1 RNA, HCV-RNA, HBsAg, and negative for antibodies to HIV 1 and 2, HCV, HTLV 1/2 and HBc by FDA licensed tests and are negative by serological test for syphilis (STS), and nonreactive for West Nile

Virus by licensed nucleic acid test (NAT).The hemoglobin is purified by an extensive processing method involving multiple chromatography steps, heat pasteurization and viral filtration.

The term “effective amount” as used herein means an amount effective and at dosages and for periods of time necessary to achieve the desired result (e.g., the enhancement of stem cell engraftment, stimulation of hematopoiesis, or myelopoiesis).

The hemoglobin can be administered at necessary doses and intervals until the desired levels of engraftment or hematopoietic cells are achieved. In one embodiment, the hemoglobin dose is from about 50 mg/kg to about 200 mg/kg, preferably 75 mg/kg to 150 mg/kg, most preferably about 100 mg/kg. The hemoglobin may be administered every 2-6 days, preferably every 4 days.

The term “animal” as used herein includes all members of the animal kingdom and is preferably a mammal including but not limited to mice, rats, rabbits, swine, nonhuman primates and humans.

In one embodiment, the animal is an immunocompromised animal such as an immunocompromised mammal that is useful in studying stem cell engraftment and human diseases. Preferably, the animal is a severe combined immunodeficient (SCID) mouse. Any strain of SCID mouse can be used including, but not limited to NSG mice NSG mouse (NOD.Cg-Prkdc^{scid} IL2rgt^{m1Wjl}/SzJ); NOG SCID (NOD/Shi-scid/IL-2Rγnull); and NOD SCID (NOD.CB17-Prkdc^{scid}/NCrCrl).

The ability of hemoglobin to enhance the engraftment of stem cells in SCID mice improves the ability to use such mice to study human hematopoiesis, to investigate human cancer pathogenesis and treatments—both for solid tumors and for leukemias and lymphomas (xenotransplants); to investigate human HIV pathogenesis and treatments; and to developing treatments for human cytopenias including neutropenias and anemias (such as cytokines, peptide and monoclonal antibodies) either as a result of stem cell defects or bone marrow injury.

In another embodiment, the animal is a human requiring a stem cell transplant. Those requiring a stem cell transplants would include patients receiving irradiation as a result of a conditioning-regime prior to a stem cell transplant (for the treatment of leukemia, for example) or those who have been subjected to radiological or chemical insult resulting in bone marrow injury.

Improved engraftment of a stem cell transplant in humans can be detected by measuring levels of CD45⁺ cells as discussed herein.

The data presented herein show that human hemoglobin can stimulate the number of human CD34⁺ cells, human CD45⁺ cells and colony-forming cells which are all indicative of the improvement of human hematopoietic engraftment and resultant increase in hematopoiesis. The stimulating hematopoiesis as a result of improved hematopoietic engraftment is useful in generating both blood cells and cells of the immune system including erythrocytes, myeloid cells (such as monocytes, macrophages, eosinophils, neutrophils, basophils and megakaryocytes) and lymphoid cells (B cells, T cells and NK cells), as well as dendritic cells of both myeloid and lymphoid origin. Stimulating hematopoiesis is useful in treating a wide range of conditions, including for use in transplant patients or patients with a bone marrow injury or disease, for treating cytopenias as well as in stimulating the development of blood cells for use in transplantation or stimulating cells of the immune system for use in treating immune deficiencies.

Accordingly, the present disclosure provides a method of stimulating hematopoiesis comprising administering an effective amount of hemoglobin to a cell or an animal in need thereof following stem cell transplantation.

The term “a cell” as used herein includes a single cell as well as a plurality or population of cells. Administering a substance to a cell includes both in vitro and in vivo administrations.

The phrase “stimulate hematopoiesis” as used herein means that the hemoglobin can stimulate or enhance the growth, proliferation, differentiation and/or mobilization of a hematopoietic stem cell or a hematopoietic progenitor cell (such as an erythroid, myeloid or lymphoid progenitor) as compared to the growth, proliferation, differentiation and/or mobilization of a hematopoietic stem cell or progenitor cell in the absence of hemoglobin.

In another embodiment, the present disclosure provides a method of stimulating myelopoiesis comprising administering an effective amount of hemoglobin to a cell or animal in need thereof following stem cell transplantation.

The phrase “stimulate myelopoiesis” as used herein means that the hemoglobin can stimulate or enhance the growth, proliferation, differentiation and/or mobilization of a myeloid cell or a myeloid progenitor cell or a stem cell as compared to the growth, proliferation, differentiation and/or mobilization of a myeloid cell or a myeloid progenitor cell or a stem cell in the absence of the hemoglobin. One skilled in the art can determine whether or not the hemoglobin can stimulate myeloid cells. For example, a colony forming assay can be used.

Stimulating the growth, proliferation, differentiation and/or mobilization of myeloid cells can be used to treat neutropenias. Stimulation of myeloid cells through hemoglobin may replace/augment the effectiveness of growth factors in overcoming neutropenias associated with bone marrow transplants with the added benefit of fewer side effects. The method can additionally be used to treat neutropenias associated with bone marrow injury, AIDS or severe congenital neutropenias. Accordingly, in a specific embodiment, the present disclosure provides a method of treating a neutropenia comprising administering an effective amount of hemoglobin to a cell or animal in need thereof.

As used herein, and as well understood in the art, “to treat” or “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The disclosure also includes a pharmaceutical composition comprising an effective amount of hemoglobin in admixture with a suitable diluent or carrier.

The pharmaceutical composition can be prepared by per se known methods for the preparation of pharmaceutically acceptable compositions that can be administered to subjects, such that an effective quantity of the hemoglobin is combined in a mixture with a pharmaceutically acceptable vehicle. Suitable vehicles are described, for example, in Remington's Pharmaceutical Sciences (Remington's Pharmaceutical Sciences, 20th ed., Mack Publishing Company, Easton, Pa., USA, 2000). On this basis, the compositions include, albeit not exclusively, solutions of the hemoglobin in association with one or more pharmaceutically acceptable vehicles or diluents, and contained in buffered solutions with a suitable pH and iso-osmotic with the physiological fluids.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. The hemoglobin may be supplied, for example but not by way of limitation, as a lyophilized powder which is reconstituted with sterile water or saline prior to administration to the patient.

Pharmaceutical compositions of the present application may comprise a pharmaceutically acceptable carrier. Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N, N, N-trimethylammonium chloride (DOTMA), diolesylphosphotidyl-ethanolamine (DOPE), and liposomes. Such compositions should contain a therapeutically effective amount of the hemoglobin together with a suitable amount of carrier so as to provide the form for direct administration to the patient.

The pharmaceutical compositions may include additional active agents useful in the stimulation of hematopoiesis including cytokines such as GM-CSF, G-CSF or Epo.

The following non-limiting example is illustrative of the present disclosure:

### EXAMPLE 1

HuSCID NSG mice were transplanted with human CD34⁺ cells enriched from 16-24 week old aborted fetal human cells. CD34⁺ cells were enriched using a Miltenyi CD34 MicroBead Kit. Animals were tested 8-14 weeks post transplant to determine the degree of human engraftment by measuring the percent of human CD45⁺ cells in the peripheral blood. Animals with greater that 30% human CD45⁺ cells in the peripheral blood were administered either PBS (control) or 1 mg/ml Hb (2 mg/animal) on days 0, 4, 8 and 12 of the study.

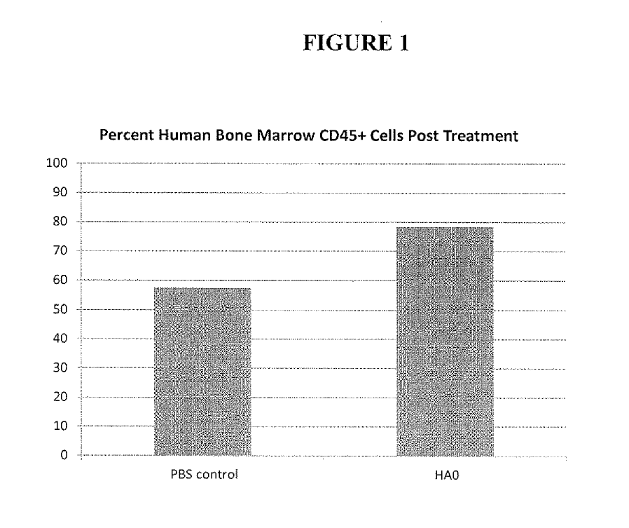

Animals were euthanized on day 14 and cells from the bone marrow were subjected to analysis by flow cytometry and were also plated in hematopoietic colony-forming assays. As shown in FIGS. 1-3, repeat hemoglobin administration increased human cell engraftment and the number of bone marrow progenitor cell colonies detected.

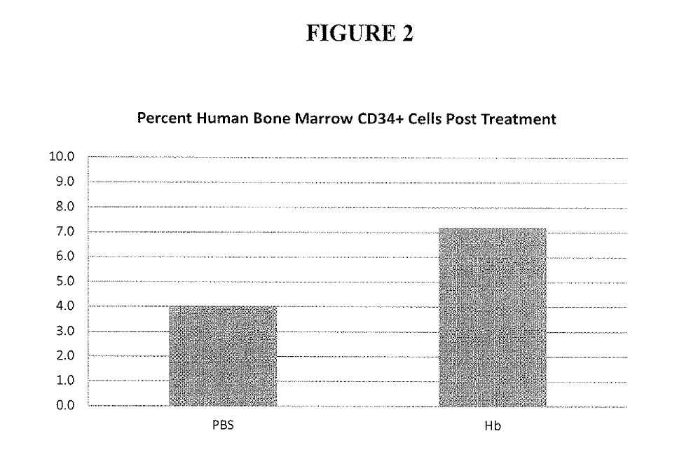

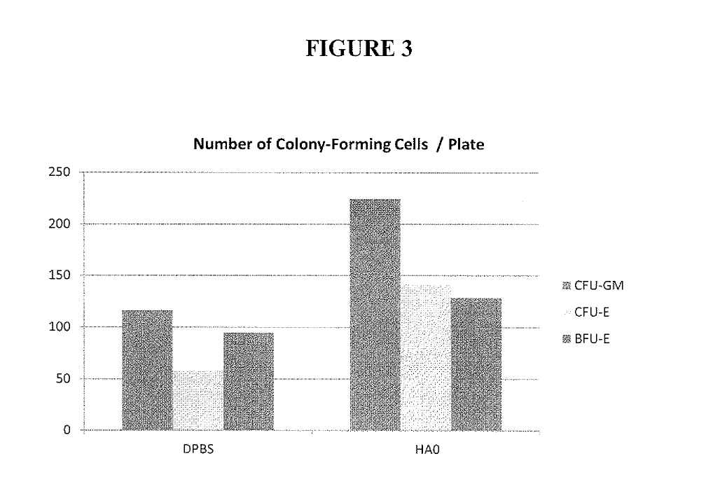

The increase in human hematopoietic cell engraftment in these animals is demonstrated the by the increased in the percentage of human CD45⁺ (FIG. 1) and human CD34⁺ cells (FIG. 2) in the bone marrow of NSG mice treated with hemoglobin (Hb). FIG. 3 demonstrates the number of hematopoietic colonies (CFUe, BFUe and CFU-GM) enumerated from the plating of human CD34⁺bone marrow cells in semi-solid colony-forming assays. CFU-GM (colony-forming unit-granulocyte-macrophage) is a colony formed from a human hematopoietic progenitor cell that is committed to the myeloid lineage. Both BFUe and CFUe are derived from progenitor cells that are committed to the erythroid lineage, with those that form BFUe less mature than those that form CFUe. While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

We claim:

1. A method of improving the engraftment of a stem cell in an animal comprising administering an effective amount of hemoglobin to the animal.

2. The method according to claim 1 wherein the animal is a SCID mouse.

3. The method according to claim 1 wherein the animal is a human.

4. The method according to claim 3 for the stimulation of hematopoiesis.

5. The method according to claim 4 for the stimulation of myelopoiesis.

6. The method according to claim 5 for the treatment of a neutropenia.

7. The method according to claim 3 for the treatment of a transplant patient.

8. The method according to claim 3 for the treatment of a patient with a bone marrow injury or disease.

9. The method according to claim 1 wherein the stem cell is a CD34⁺ cell.

10. The method according to claim 1 further comprising transplanting stem cells in the animal prior to or concurrently with the administering of hemoglobin.

11. The method according to claim 10 wherein the hemoglobin is administered 1 to 10 days after the stem cell transplant.

12. The method according to claim 1 wherein improved engraftment is detected by measuring levels of CD45⁺ cells.

13. The method of claim 12 wherein the levels of CD45⁺ cells are at least 10% higher than the levels in a control animal that did not receive hemoglobin treatment.

14. The method of claim 12 wherein the levels of CD45⁺ cells are at least 30% higher than the levels in a control animal that did not receive hemoglobin treatment.
